(19) United States
(12) Patent Application Publication
(10) Pub. No.: US 2005/0149181 A1
(43) Pub. Date: Jul. 7, 2005

(54) Bileaflet prosthetic valve and method of manufacture

(75) Inventor: Carol E. Eberhardt, Fullerton, CA (US)

Correspondence Address: DICKE, BILLIG & CZAJA, PLLC, Fifth Street Towers, Suite 2250, 100 South Fifth Street, Minneapolis, MN, 55402, US

(73) Assignee: Medtronic, Inc.

(21) Appl. No.: 10/752,864

(22) Filed: Jan. 7, 2004

(51) Int. Cl. A61F 2/24

(52) U.S. Cl. 623/2.14; 623/2.16

(57) ABSTRACT

A prosthetic valve including a body, a first leaflet, and a second leaflet. The first leaflet extends across and is coupled to the body. The first leaflet is cut from a first porcine aortic valve and defines a first inner surface. The second leaflet extends across and is coupled to the body opposite the first leaflet. The second leaflet is cut from a second porcine aortic valve and defines a second inner surface.

### BACKGROUND OF THE INVENTION

The present invention relates to an implantable prosthetic valve. More particularly, the present invention relates to a bileaflet implantable prosthetic valve with redundant coaptation to be implanted during heart valve replacement surgery.

There are four valves of the heart, the mitral valve, the aortic valve, the tricuspid valve, and the pulmonary valve. Anatomically and generally speaking, each valve forms or defines a valve annulus and valve leaflets. Although similar in general function, the mitral valve differs significantly in anatomy from the other valves, in particular, the aortic valve. The annulus of the mitral valve is somewhat “D” shaped or elongated whereas the annulus of the aortic valve is more nearly circular. Furthermore, the mitral valve includes two leaflets that are oval or “D” shaped, in contrast to the aortic valve, which includes three leaflets that are more nearly circular. Mitral valves are also subject to higher pressure and longer closure periods than are aortic valves.

To accommodate such conditions, native mitral valves incorporate redundant coaptation. The term “redundant coaptation” is used to refer to closure of the valve at more than one line of interaction between the leaflets. In particular, the native mitral valve leaflets interact during closure tightly mating or coapting along a first line. In addition, the native mitral valve leaflets also interact or coapt at multiple points between the first line and the free edges of the leaflets (i.e., the edges of the leaflets not attached to the remaining valve). Moreover, the native mitral valve leaflets, close to interact or coapt with one another such that the free edges are gathered or puckered rather than held substantially taut. The repetitious or redundant coaptation bolsters the integrity of the valve to better maintain closure during relatively long periods and to better withstand the high closure pressures.

Any heart valve can be subjected to or incur damage that requires the valve to be repaired or replaced. A majority of patients with heart valve disease undergo heart valve replacement surgery rather than heart valve repair. Various types and configurations of prosthetic heart valves are used to replace diseased, human heart valves. In general terms, the prosthetic heart valve design attempts to replicate the function of the valve being replaced and thus will include valve or leaflet-like structures. With this in mind, prosthetic heart valves are generally classified as either forming relatively rigid leaflets or forming relatively flexible leaflets. The category including prosthetic heart valves which form relatively flexible leaflets includes bioprosthetic heart valves having leaflets made of a biological material as well as prosthetic heart valves having leaflets made of synthetic (e.g., polymeric) material. Flexible leaflet prosthetic heart valves are generally categorized as having a frame or a stent or as having no stent.

Despite the different anatomies of the different heart valves described above, conventional, flexible leaflet, prosthetic heart valves designed for use with the different heart valves are surprisingly similar. In particular, in creating flexible leaflet, prosthetic heart valves using porcine tissue for leaflets, the porcine aortic valve is typically used to make both the aortic and mitral prosthetic valves. More commonly, a single type of prosthetic porcine valve is manufactured and used for replacement of both the aortic and mitral valves. The aortic porcine valve is circular, similar to the native human aortic valve. However, as previously described, the native human mitral valve is more oval or elongated than circular. Therefore, during implantation, the typical mitral valve prosthetic made from a porcine aortic valve must be forced to conform to the non-circular annulus of the native mitral valve.

In addition to the different overall valve shapes, a porcine aortic valve and the resulting prosthetic valves each have three leaflets while a native mitral valve has only two leaflets. Moreover, the conventional tri-leaflet prosthetic valves do not incorporate redundant coaptation while closed and, therefore, such prosthetic valves are not specifically designed to withstand the higher pressures and longer closure periods experienced by the mitral valve. As such, the anatomy of the prosthetic valves typically used to replace a mitral valve do not sufficiently replicate the native mitral valve anatomy.

More recently, flexible leaflet, prosthetic valves have been developed incorporating the bileaflet anatomy of the native mitral valve. In particular, FIGS. 1A and 1B illustrate a prior art bileaflet, prosthetic valve generally at 10. The conventional prosthetic valve 10 includes a stent 12 (generally indicated), a first leaflet 14, and a second leaflet 16. The stent 12 defines an annular ring 18, a first strut 20, and a second strut 22. The first strut 20 is coupled with and extends from the annular ring 18 to form a rounded tip 24. The second strut 22 is diametrically opposed to the first strut 20 and is coupled with and extends from the annular ring 18 to form a rounded tip 26.

The first leaflet 14 is coupled with the stent 12 by suturing the first leaflet 14 to the annular ring 18 and the first and second struts 20 and 22. As such, the first leaflet 14 extends between the struts 20 and 22 to define a free edge 30 opposite the annular ring 18. Similarly, the second leaflet 16 is coupled with the stent 12 by suturing the second leaflet 16 to the annular ring 18 and the struts 20 and 22. Therefore, the second leaflet 16 extends between the struts 20 and 22 opposite the first leaflet 14 to define a free edge 32 opposite the annular ring 18.

As illustrated in FIG. 1A, the prosthetic valve 10 closes such that the free edge 30 and the free edge 32 coapt or fit together to tightly close the prosthetic valve 10. In particular, the free edges 30 and 32 directly abut one another in the closed position. Notably, the intersection between the free edges 30 and 32 defines a catenary 34 between the first tip 24 of the first strut 20 and the second tip 26 of the second strut 22. The catenary 34 is more precisely an imaginary curve that extends between and, in effect, hangs from, the first tip 24 and the second tip 26. In the case of the prosthetic valve 10, the catenary 34 represents the first and only line of interaction between the first and second leaflets 14 and 16 during closure. When in the closed position, the first leaflet 14 and the second leaflet 16 are each maintained in a relatively taut manner.

As illustrated by comparison of FIGS. 1A and 1B, to open the prosthetic valve 10, the free edge 30 of the first leaflet 14 transitions away from the catenary 34 in a direction opposite the free edge 32 of the second leaflet 16. Simultaneously, the free edge 32 of the second leaflet 16 transitions away from the catenary 34 in a direction opposite the free end 30. Accordingly, when in an open position, the prosthetic valve 10 forms an open cavity for blood to flow through. Notably, upon opening (FIG. 1B), each of the free edges 30 and 32 has a length equal to the length of the catenary 34 (FIG. 1A). Accordingly, upon opening, the prosthetic mitral valve 10, more particularly the free edges 30 and 32, form an opening 36 having a perimeter substantially equal to twice the length of the catenary 34. As such, the length of the catenary 34 limits the size of the opening 36, which may impede blood flow through the valve prosthetic 10.

Conventional flexible leaflet, prosthetic valves having no stent typically are tri-leaflet valves that tightly coapt such that the free edges of each leaflet abut one another upon closure of the stentless valve. Often, an entirety (i.e., the valve annulus and leaflets) of a porcine aortic valve is harvested, treated, and used as the replacement valve in heat valve replacement surgery. However, similar to the conventional stented valves, conventional stentless valves are not constructed or modified to withstand relatively high pressures and prolonged closing intervals.

As described above, upon closure, the leaflets of a typical prosthetic valves are maintained in a relatively taut manner. The taut leaflets are in contrast to the puckered leaflets of the native mitral valve, which provide for redundant coaptation, a stronger valve closure, and a larger valve opening. As such, a need exists for a prosthetic valve that provides for a stronger valve closure and for a larger valve opening. In particular, a need exists for a prosthetic valve that is more adept to high pressures and prolonged closing times.

### SUMMARY OF THE INVENTION

One aspect of the present invention relates to a prosthetic valve including a body, a first leaflet, and a second leaflet. The first leaflet extends across and is coupled to the body. The first leaflet is cut from a first porcine aortic valve and defines a first inner surface. The second leaflet extends across and is coupled to the body opposite the first leaflet. The second leaflet is cut from a second porcine aortic valve and defines a second inner surface.

Another aspect of the present invention relates to a prosthetic valve including a body, a first leaflet, and a second leaflet. The first leaflet extends across and is sutured to the body. The first leaflet has an elongated shape. The second leaflet extends across and is sutured to the body opposite the first leaflet. The second leaflet has an elongated shape.

Another aspect of the present invention relates to a prosthetic valve including a body, a first leaflet, and a second leaflet. The first leaflet extends across and is sutured to the body. The first leaflet is cut from a first porcine aortic valve, defines a first inner surface, and has an elongated shape. The second leaflet extends across and is sutured to the body opposite the first leaflet. The second leaflet is cut from a second porcine aortic valve, defines a second inner surface, and has an elongated shape.

Yet another aspect of the present invention relates to a method of manufacturing a prosthetic mitral valve. The method includes providing a body, cutting a first leaflet defining a first inner surface from a first porcine aortic valve, coupling the first leaflet to the body, cutting a second leaflet defining a second inner surface from a second porcine aortic valve, and coupling the second leaflet to the body opposite the first leaflet.

### BRIEF DESCRIPTION OF THE DRAWINGS

FIG. 1A is a perspective view of a prior art prosthetic valve in a closed position;

FIG. 1B is a perspective view of the prior art prosthetic valve illustrated in FIG. 1A in an open position;

FIG. 2 is a perspective view of one embodiment of a bileaflet prosthetic valve in a closed position in accordance with the present invention;

FIG. 3 is a perspective view of the bileaflet prosthetic valve illustrated in FIG. 2 in an opened position;

FIG. 4 is a perspective view of one embodiment of a stent and a cloth covering of the bileaflet prosthetic valve illustrated in FIG. 2;

FIG. 5A is a schematic view of one embodiment of a left cusp of a porcine aortic valve for use in the bileaflet prosthetic valve illustrated in FIG. 2;

FIG. 5B is a schematic view of one embodiment of another left cusp of a porcine aortic valve for use in the bileaflet prosthetic valve illustrated in FIG. 2;

FIG. 6 is a perspective view of one embodiment of a stentless, bileaflet prosthetic valve according to the present invention; and

FIG. 7 is a top view of the stentless, bileaflet prosthetic valve of FIG. 6.

### DETAILED DESCRIPTION OF THE PREFERRED EMBODIMENTS

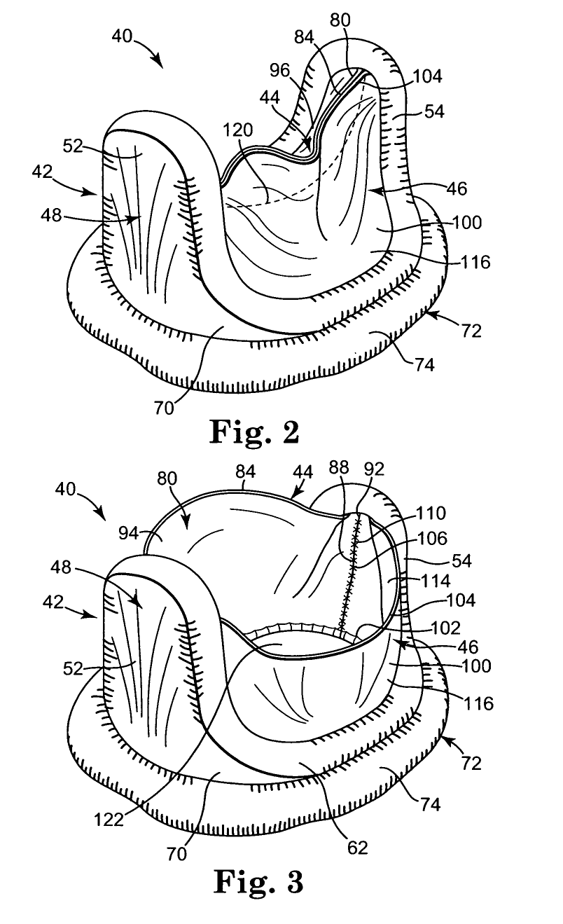

One preferred embodiment of a bileaflet, prosthetic valve 40 in accordance with the present invention is illustrated in FIGS. 2 and 3. The prosthetic valve 40 includes a body 42, a first leaflet 44, and a second leaflet 46. The body 42 serves as the support structure to which the first leaflet 44 and the second leaflet 46 are opposingly attached. In particular, the leaflets 44 and 46 are attached such that in a closed position, as illustrated in FIG. 2, the first leaflet 44 interacts with the second leaflet 46 to close the prosthetic valve 40. More precisely, the first leaflet 44 and the second leaflet 46 redundantly coapt to close and to prevent blood flow through the prosthetic valve 40 prosthetic valve 40. When open, as illustrated in FIG. 3, the first leaflet 44 and the second leaflet 46 are pulled away from one another, thereby opening the prosthetic valve 40 to allow blood flow to freely pass through the prosthetic valve 40.

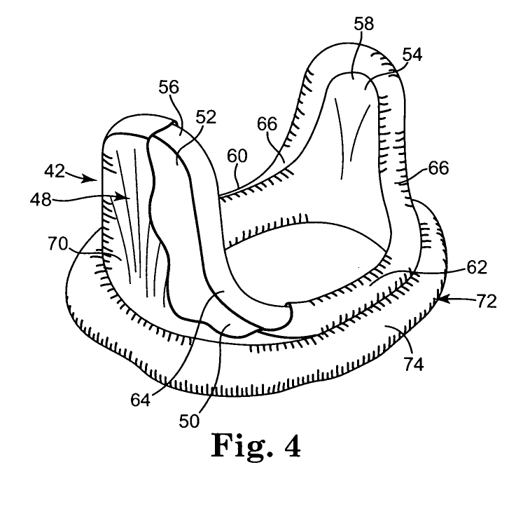

As illustrated in FIG. 4, in one embodiment, the body 42 is a stent 48 including an annular ring 50, a first strut 52, and a second strut 54 (generally indicated). The annular ring 50 acts as a base member to which the struts 52 and 54 are attached or otherwise extend from. Although the annular ring 50 may be formed with a circular shape, in one embodiment, the preferred shape of the annular ring 50 is parabolic to more closely mimic the native mitral valve. The first strut 52 extends from the annular ring 50 to a first rounded extremity or tip 56. Similarly, the second strut 54 is diametrically opposed to the first strut 52 and extends from the annular ring 50 to a second rounded extremity or tip 58. The annular ring 50 defines a first relief 60 (generally indicated) between the struts 52 and 54 and a second relief 62 (generally indicated) between the struts 52 and 54 opposite the first relief 60. Each relief 60 and 62 defines opposing smooth curves 64 and 66, respectively, adjacent to the respective struts 52 and 54 such that the reliefs 60 and 62 are each substantially arcuate in shape.

Although the struts 52 and 54 are depicted as being diametrically opposed, in other embodiments, the struts 52 and 54 are slightly offset from being truly diametrically opposed to one another (i.e., the second strut 54 is nonsymmetrically positioned relative to the first strut 52). In such an embodiment, the first relief 60 has a longer length than the second relief 62 (or vice-versa) and later attachment utilizes a first leaflet 44 (FIG. 3) being slightly larger than the second leaflet 46 (FIG. 3). In one embodiment, the differently sized leaflets 44 and 46 further mimic the natural sizing of native mitral valve leaflets.

In one embodiment, the stent 48 is formed as an integral and homogeneous unit. In an alternative embodiment, the stent 48 is made of discrete pieces subsequently joined together. Preferably, the stent 48 is made as slim and light as is compatible with the needed strength of the prosthetic valve 40 (FIG. 2) and to avoid the creation of sharp edges. In one embodiment, the annular ring 50 and the struts 52 and 54 are made of a slightly flexible, elastomeric material such as a synthetic plastic material including but not limited to polypropylene or acetal copolymer. In another embodiment, the annular ring 50 and the struts 52 and 54 are formed of a thin wire or contoured thermoplastic material, e.g., polypropylene, celcon, or acetyl homopolymer. In one embodiment, the annular ring 50 and the struts 52 and 54 are formed of a metal material including, but not limited to, Eligiloy®, stainless steel, nitinol®, etc. Preferably, the struts 52 and 54 are formed of stiff but resiliently bendable material which allows the rounded extremities 56 and 58 of the struts 52 and 54 to deflect inward upon application of an external force, such as the force of a holder (not shown) used to insert the prosthetic valve 40 into the heart valve annulus. Upon removal of the external force, the struts 52 and 54 are adapted to return to the original position as illustrated in FIG. 4.

Preferably, the stent 48 further includes a cloth covering 70, which covers and is sutured to and around the annular ring 50 and the struts 52 and 54. In one embodiment, the annular ring 50 and the struts 52 and 54 each defines one or a plurality of apertures (not shown) to facilitate suturing the covering 70 to the annular ring 50 and the struts 52 and 54. The covering 70 is preferably formed of a biocompatible, fabric material. In one embodiment, the covering 70 is a porous, woven or knitted polytetrafluoroethylene (such as that sold under the tradename Teflon®) or polyester (such as that sold under the tradename Dacron®).

In one embodiment, a suture ring 72 is coupled with the stent 48 to facilitate subsequent suturing of the prosthetic valve 40 to a heart valve annulus (not shown). The suture ring 72 is formed of a tubular cloth covering 74, which is similar to the cloth covering 70 attached to the stent 48. The cloth covering 74 is sutured to the cloth covering 70 of the stent 48 about the outer perimeter of the annular ring 50 opposite the extension of the struts 52 and 54. In one embodiment, the suture ring 72 further includes biocompatible cushion or stuffing material (not shown) disposed within the tubular cloth covering 74. In one embodiment, the suture ring 72 further includes an additional support ring (not shown) disposed within the cloth covering 74 to provide additional support to the prosthetic valve 40.

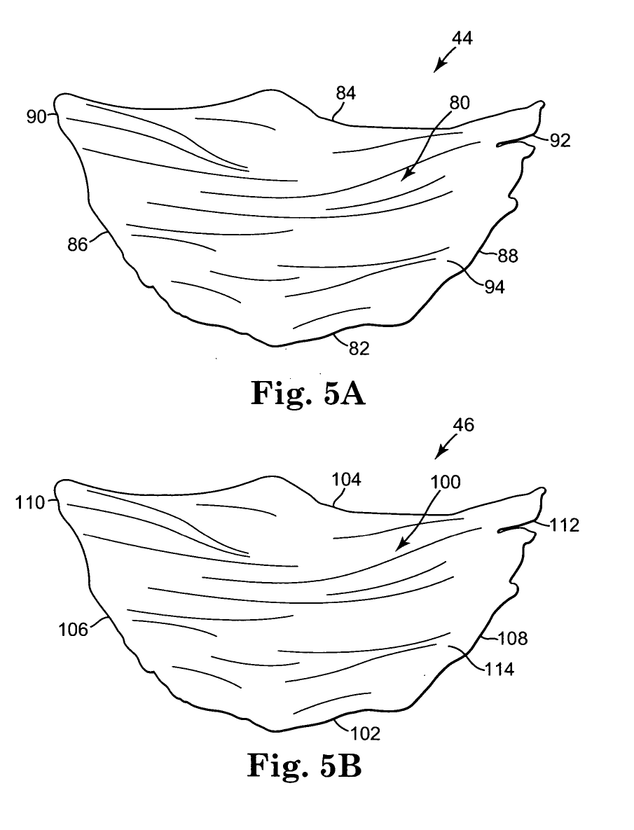

FIG. 5A illustrates one embodiment of the first leaflet 44. Preferably, first leaflet 44 is a first left cusp 80, which is cut from a porcine aortic valve (not shown). In one embodiment, the left cusp 80 is cut from a porcine aortic valve examined and found inadequate for use in or as an aortic valve prosthesis. As such, the left cusp 80 can be cut from a porcine aortic valve that was otherwise rejected for possible use as an aortic valve prosthesis. In particular, upon selection of a left cusp 80 for use in the prosthetic valve 40, the selected left cusp 80 is treated to fix and sterilize the valve tissue as well as to decrease the antigenicity of the left cusp 80. In one embodiment, the left cusp 80 undergoes cross-linking using glutaraldehyde. However, in other embodiments, alternative chemistries are used to cross-link the first left cusp 80. After treatment, the left cusp 80 is cut from the remainder of a first porcine aortic valve for use in the prosthetic valve 40, resulting in the first leaflet 44.

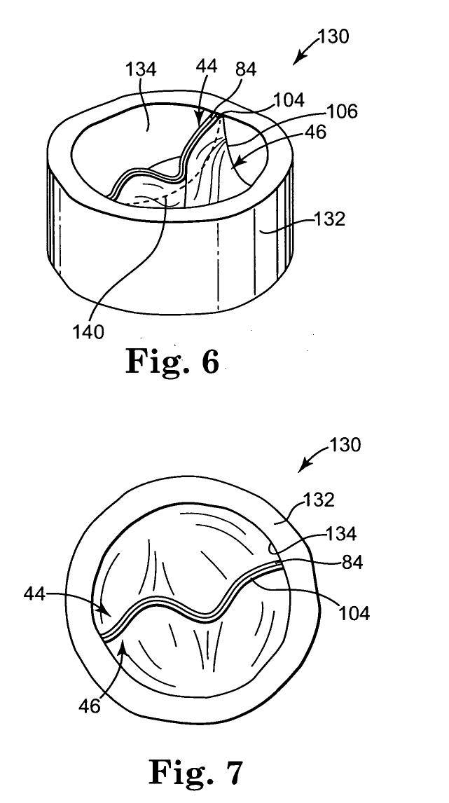

The first leaflet 44 is elongated or generally “D” shaped and defines a cut edge 82, a free edge 84, a first attachment edge 86, and a second attachment edge 88. The cut edge 82 was formally attached to and part of the first porcine aortic valve (not shown), and was cut in harvest of the first left cusp 80 from the first porcine aortic valve. The free edge 84 is opposite the cut edge 82. As part of the porcine aortic valve, the free edge 84 was unattached and free to periodically coapt with the other aortic cusps (not shown). The first and second attachment edges 86 and 88 run between the cut edge 82 and the free edge 84 opposite one another, and were also cut in harvest of the first left cusp 80 from the first porcine aortic valve. The first attachment edge 86 further defines a first commissure portion 90 near the free edge 84. Similarly, the second attachment edge 88 defines a second commissure portion 92 near the free edge 84. The first leaflet 44 defines an inner surface 94 and an outer surface 96 (FIGS. 2 and 6) opposite the inner surface 94.

As illustrated in FIG. 5B, the second leaflet 46 is preferably a second left cusp 100, which is similar to the first left cusp 80 described above. In particular, the second left cusp 100 is cut from the remainder of a second porcine aortic valve (not shown). Further, the second left cusp 100 is treated to fix and sterilize the tissue as well as to decrease the antigenicity of the second left cusp 100 as described above with respect to the first leaflet 44 (FIG. 5A). The second leaflet 46 is elongated or generally “D” shaped and defines a cut edge 102, a free edge 104, a first attachment edge 106, and a second attachment edge 108 similar to the cut edge 82, the free edge 84, the first attachment edge 86, and the second attachment edge 88 of the first leaflet 44, respectively. The first attachment edge 106 defines a first commissure portion 110 near the free edge 104. Accordingly, the second attachment edge 108 defines a second commissure portion 112 near the free edge 104. The second leaflet 46 defines an inner surface 114 and an outer surface 116 (FIG. 2) opposite the inner surface 114.

Preferably, the first leaflet 44 and the second leaflet 46 are substantially similar in size. In one embodiment, the first leaflet 44 is slightly larger than the second leaflet 46. In alternative embodiments, the leaflets 44 and 46 are formed of other tissue, such as porcine, bovine, or human pericardium, fascia lata, and dura mater. In such embodiments, the leaflets 44 and 46 are, however, formed or cut from the tissue to define elongated or “D” shapes similar to the shape of the first and second left cusps 80 and 100 described above, rather than the typical circular leaflet shape.

As illustrated in FIG. 3, during manufacture, the cut edge 102, the first attachment edge 106, and the second attachment edge 108 (FIG. 5B) of the selected second leaflet 46 are all sutured to the stent 48. In particular, the second leaflet 46 is substantially centered with respect to the second relief 62 of the annular ring 50. The cut edge 102 of second leaflet 46 is sutured to the covering 70 of the annular ring 50 at or below the second relief 62. The first attachment edge 106 extends along and is sutured to the covering 70 over the interior side of the second strut 54. In one embodiment, the first attachment edge 106 is sutured to the second strut 54 such that the first commissure portion 110 is positioned substantially on a vertical centerline of the second strut 54. Although not illustrated, the second attachment edge 108 similarly extends along and is sutured to the first strut 52. In one embodiment, the second attachment edge 108 is sutured to the covering 70 over the interior side of the first strut 52 such that the second commissure portion 112 (FIG. 5B) is positioned substantially on the vertical centerline of the first strut 52. As such, second leaflet 46 is attached to the stent 48 on all edges 102, 106, and 108 but the free edge 104.

The free edge 104 remains unsutured and extends between the extremities 56 and 58 of the struts 52 and 54. As such, the free edge 104 can freely transition between an open and a closed position. In particular, when in the closed position, the free edge 104 hangs near but above a catenary 120 defined between the extremities 56 and 58 of the struts 52 and 54. The catenary 120 is an invisible curve representing the line of interaction between the leaflets 44 and 46 nearest the annular frame 50. Notably, the free edge 104 of the second leaflet 46 has a length that is longer than a length of the catenary 120 between extremities 56 and 58. When in the open position, as best illustrated in FIG. 3, the free edge 104 extends from the annular ring 50 in a substantially semi-annular manner.

During manufacture, the cut edge 82, the first attachment edge 86 (FIG. 5A), and the second attachment edge 88 of the first left leaflet 44 are sutured to the stent 48 of the prosthetic valve 40. In particular, the first leaflet 44 is substantially centered with respect to the first relief 60 (FIG. 4) of the annular ring 50 as described and illustrated with respect to the second leaflet 46 and second relief 62. The cut edge 82 and is sutured to the covering 70 at or below the first relief 60. Although not fully illustrated, the first attachment edge 86 extends along and is sutured to the covering 70 over the interior side of the first strut 52 in a similar manner as described for second attachment edge 108.

In one embodiment, the first attachment edge 86 is sutured to the first strut 52 such that the first commissure portion 90 is positioned substantially on the vertical centerline of the first strut 52. The second attachment edge 88 extends along and is sutured to the covering 70 over the interior side of the second strut 54. In one embodiment, the second attachment edge 88 is sutured to the second strut 54 such that the second commissure portion 92 is positioned substantially on the vertical centerline of the second strut 54. As such the first leaflet 44 is attached to the stent 48 on all the edges 82, 86, and 88 but the free edge 84.

In a preferred embodiment, the first leaflet 44 and the second leaflet 46 are sutured to the first strut 52 such that the second commissure portion 92 of the sutured first leaflet 44 is positioned adjacent to the first commissure portion 110 of the sutured second leaflet 46. In one embodiment, the first leaflet 44 and the second leaflet 46 are sutured to the first strut 52 such that the attachment edges 86 and 108 of the leaflets 44 and 46 are only positioned adjacent one another along the second commissure portion 92 of the first leaflet 44 and the first commissure portion 110 of the second leaflet 46. Similarly although hidden in FIG. 3, in a preferred embodiment, the first commissure portion 90 (FIG. 5A) of the sutured first leaflet 44 is positioned on the second strut 54 adjacent to the second commissure portion 112 (FIG. 5B) of the sutured second leaflet 46. Notably, other variations of suturing the leaflets 44 and 46 to the first and second struts 52 and 54 will be apparent to those of ordinary skill in the art.

The free edge 84 remains unsutured and extends between the extremities 56 and 58 of the struts 52 and 54. As such, the free edge 84 can freely transition between an open and a closed position. In particular, when in the closed position, the free edge 84 hangs near but above the catenary 120 defined between the extremities 56 and 58 of the struts 52 and 54 as best illustrated in FIG. 2. Notably, the free edge 84 of the first leaflet 44 has a length, which is longer than a length of the catenary 120 between the extremities 56 and 58. When in the open position, illustrated in FIG. 3, the free edge 84 extends from the annular ring 50 in a substantially semi-annular manner.

Upon assembly, the leaflets 44 and 46 are positioned and tightly and substantially continuously sutured to the stent 48 such that all seams or connections points between the leaflets 44 and 46 and the stent 48 substantially prevent blood flow from traveling through or escaping from the seams. Preferably, upon assembly, no blood flow escapes or passes through a properly implanted prosthetic valve 40 in the closed position.

Following assembly, when the prosthetic valve 40 is in the closed position (FIG. 2), the inner surfaces 94 and 114 (FIG. 3) of the first leaflet 44 and the second leaflet 46, respectively, interact or more precisely coapt with one another along and above the catenary 120. However, the free edge 84 of the first leaflet 44 and the free edge 104 of the second leaflet 46 are not held taut near the catenary 120, nor do the free edge 84 and the free edge 104 mate directly with one another. Rather, due to the excess tissue of each of the leaflets 44 and 46 and the fact that each of the free edges 84 and 104 has a length longer than the length of the catenary 120, upon closing, each of the free edges 84 and 104 is slightly puckered or gathered.

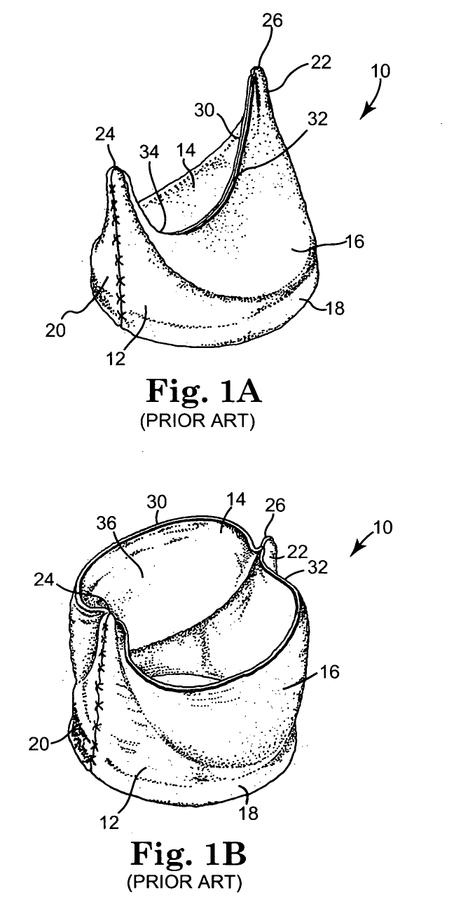

Further due to the extra tissue of each leaflet 44 and 46, as compared to the prior art, the first inner surface 94 and the second inner surface 114 redundantly coapt, or tightly interact to close about the catenary 120 and at a plurality of areas between the catenary 120 and the free edges 84 and 104. As such, substantial portions of the inner surface 94 of the first leaflet 44 and the inner surface 114 of the second leaflet 46 between the portion that coapts about the catenary 120 and the free edges 84 and 104 interact to form an enhanced area interface as compared to prior art leaflets that coapt only along a single catenary (see FIGS. 1A and 1B). Notably, the redundant coaptation of, or repetitious interaction between, the leaflets 44 and 46 increases the integrity of the closure of the bileaflet, prosthetic valve 40. The redundant coaptation not only mimics the native mitral valve, but also provides a robust seal between the two leaflets 44 and 46 during closure, to prevent leakage through the prosthetic valve 40 during closure. Moreover, the benefit of the additional closure integrity is increased due to the prolonged closure periods and the relatively high pressures to be experienced by the prosthetic valve 40 upon implant within a patient.

Upon transition to an open position, and as best illustrated in FIG. 3, the free edge 84 and the free edge 104 transition away from the catenary 120, opposite one another. When open, the free edges 84 and 104 each extend from the annular ring 50 in a semi-annular manner such that the prosthetic valve 40 merely forms a substantially tubular cavity for blood flow to travel through. Notably, as mentioned above, the length of the first free edge 84 is longer than the length of the catenary 120. Similarly, the length of the second free edge 104 is greater than the length of the catenary 120. As such, upon opening of the prosthetic valve 40, an opening 122 is formed having a perimeter substantially equal to the sum of the length of the first free edge 84 and the length of the second free edge 104. Otherwise stated, the opening 122 is formed having a perimeter greater than double the length of the catenary 120. The relatively large opening, as compared to the opening of the prior art prosthetic mitral valves, allows blood to flow through the prosthetic valve 40 with a lessened degree of obstruction.

The prosthetic valve 40 can be manufactured in a plurality of sizes to provide replacement valves for the plurality of annulus sizes found in heart valve replacement patients. In one embodiment, the prosthetic valve 40 is manufactured in a plurality of sizes to provide replacement valves for mitral valves, aortic valves, tricuspid valves, and pulmonary valves. In one embodiment, the maximum diameter of the bileaflet prosthetic mitral valve range from approximately 25 mm to 35 mm. As such, prior to attachment, a first left cusp 80 and a second left cusp 100 are selected to correspond with the size of the particular stent 48 of the prosthetic valve 40 being manufactured.

During use, the prosthetic valve 40 is implanted and sutured to the heart valve annulus of the mitral valve (not shown). In particular, a surgeon sutures the suture ring 72 to the annulus ledge or within the annulus opening depending upon the implantation technique (intra-annular or supra-annular) being utilized for the particular heart valve replacement surgery. In one embodiment, the prosthetic valve 40 is implanted through a catheter. Notably, the two leaflet nature of the prosthetic valve 40 may make the prosthetic valve 40 more compressible and, therefore, even more conducive to catheter implantation than its three leaflet counterparts. In other embodiments, the prosthetic valve 40 is implanted without the use of a catheter. The prosthetic valve 40 is a bileaflet valve that opens widely and closes incorporating redundant coaptation in a manner similar to the native mitral valve. Although described as replacing a mitral valve, the prosthetic valve 40 can be used in valve replacement surgery for an aortic valve, a tricuspid valve, or a pulmonary valve.

FIGS. 6 and 7 illustrate another embodiment of a bileaflet prosthetic valve generally indicated at 130. The prosthetic valve 130 includes a body 132, the first leaflet 44, and the second leaflet 46. The body 132 is tubular and, in one embodiment, is round or parabolic (i.e., elongated) in shape. In one embodiment, the tubular body 132 is formed of one of the following: a porcine tissue, a pericardial tissue, a venous material, a cloth, or a mesh material. In one embodiment, the tubular body 132 is a porcine aortic root.

Each of the first and second leaflets 44 and 46 are sized and selected to correspond with the size of the tubular body 132. The first and second leaflets 44 and 46 are attached to the tubular body 132 in a similar manner as leaflets 44 and 46 are attached to the stent 48. In particular, with additional reference to FIGS. 5A and 5B, the cut edge 82, the first attachment edge 86, and the second attachment edge 88 of the first leaflet 44 are all sutured to an inner surface 134 of the tubular body 132. Similarly, the cut edge 102, the first attachment edge 106, and the second attachment edge 108 of the second leaflet 46 are sutured to the inner surface 134 of the tubular body 132. The cut edges 82 and 102 are attached by suture to the inner surface 134 opposite one another and along a bottom circumference (not shown) of the inner surface 134. The attachment edges 86, 88, 106, and 108 extend away from the cut edges 82 and 102 and are sutured to the inner surface 134. In one embodiment, the leaflets 44 and 46 are sutured to the inner surface 134 such that the commissure 92 of the second edge 88 is positioned adjacent the commissure portion 110 of the first edge 106. Similarly, the leaflets 44 and 46 are sutured such that the commissure portion 90 of the first edge 86 is positioned adjacent the commissure portion 112 of the second edge 108.

The free edges 84 and 104 remain unsutured to freely transition between an open and a closed position as described above with respect to prosthetic valve 40. In particular, the leaflets 44 and 46 are configured and attached to the tubular body 132 such that the inner surfaces 94 and 114 of the leaflets 44 and 46 redundantly interact or, more precisely, coapt with one another along and above a catenary 140, which extends between the commissure portions 92 and 100 and the commissure portions 90 and 112. Notably, the free edges 84 and 104 each have a length longer than a length of the catenary 140. Upon opening the free edges 84 and 104 define an opening (not shown) that is similar to the opening 122 (FIG. 3) having a perimeter greater than double the length of the catenary 140.

The prosthetic valve 130 can be manufactured in a plurality of sizes to provide replacement valves for a plurality of annulus sizes found in heart valve replacement patients. In one embodiment, the prosthetic valve 130 is manufactured in a plurality of sizes to provide replacement valves for mitral valves, aortic valves, tricuspid valves, and pulmonary valves. The prosthetic valve 130 is implanted in a similar manner as described above with respect to the prosthetic valve 40. Normally the tubular body 132 is placed within the annulus opening (not shown) and sutured to the annulus edge or within the annulus opening depending upon the implantation technique being utilized for the particular heart valve replacement surgery.

In general, a prosthetic, bileaflet valve according to the present invention is shaped substantially similar to and substantially mimics the functioning of the native mitral valve. The bileaflet valve prosthetic includes cusps or leaflets having a longer free edge than the catenary in which they originally coapt. As such, the opening periodically formed by the bileaflet valve is not limited in size or cross-section due to the length of the catenary. Rather, the bileaflet valve of the present invention opens widely, to cause less obstruction of blood flow than prior art valve prosthetics. Less obstruction of blood flow directly correlates to increased valve durability as well as increased post-operative patient activity and overall patient well being.

In addition, the bileaflet valve of the present invention redundantly coapts similar to the native mitral valve. The redundant coaptation ensures a better seal of the closed valve, which is especially important under the relatively high pressure and long closure periods of the mitral valve. The high integrity closure prevents or decreases blood leakage through the bileaflet valve while the bileaflet valve is in the closed position. Decreasing undesired leakage of the bileaflet valve decreases complications associated with heart valve replacement surgery as well contributes to the overall well being of the patient.

Although the present invention has been described with reference to preferred embodiments, workers skilled in the art will recognize that changes can be made in form and detail without departing from the spirit and scope of the present invention.

1. A prosthetic valve comprising:
a body;
a first leaflet extending across and coupled to the body, the first leaflet being cut from a porcine aortic valve and defining a first inner surface; and
a second leaflet extending across and coupled to the body opposite the first leaflet, the second leaflet being cut from a porcine aortic valve and defining a second inner surface; wherein the prosthetic valve is configured such that upon closure of the first and second leaflets, the first inner surface and the second inner surface redundantly coapt.

2. (canceled)

3. The prosthetic valve of claim 1, wherein the first leaflet and the second leaflet are each a left cusp.

4. The prosthetic valve of claim 1, wherein the first and second leaflets each define a free edge, and upon closure of the prosthetic valve, the free edges interact and pucker.

5. The prosthetic valve of claim 1, wherein the first and second leaflets are coupled with the body in a manner characterized by a lack of tautness.

6. The prosthetic valve of claim 1, wherein the first and second leaflets each define a cut edge fixed to the body, a free edge not fixed to the body, and the first and second inner surfaces coapt with each other along a catenary spaced from the free edges, and further wherein the catenary represents a line of interaction between the first and second leaflets nearest the cut edge.

7. The prosthetic valve of claim 6, wherein upon closure of the prosthetic valve, the first inner surface and the second inner surface interact to define an enhanced surface area interface between the catenary and the free edges.

8. The prosthetic valve of claim 6, wherein each of the free edges has a length that is longer than a length of the catenary.

9. The prosthetic valve of claim 6, wherein upon opening of the first and second leaflets, the first and second free edges define an opening, the opening having a perimeter greater than twice a length of the catenary.

10. The prosthetic valve of claim 1, wherein the prosthetic valve is characterized by the absence of a third leaflet.

11. The prosthetic valve of claim 1, wherein the body includes a stent including an annular frame, a first strut extending from the annular frame, and a second strut spaced from the first strut and extending from the annular frame.

12. The prosthetic valve of claim 10, wherein the first strut and the second strut are nonsymmetrically positioned with respect to the annular frame.

13. The prosthetic valve of claim 1, wherein the prosthetic valve is a prosthetic mitral valve.

14. The prosthetic valve of claim 1, wherein the body includes a tubular body.

15. The prosthetic valve of claim 14, wherein the tubular body is an aortic root.

16. A prosthetic valve comprising:
a body;
a first leaflet extending across and sutured to the body, the first leaflet having an elongated shape and defining a cut edge sutured to the body, a free edge not sutured to the body, and an inner surface; and
a second leaflet extending across and is sutured to the body opposite the first leaflet, the second leaflet having an elongated shape and defining a cut edge sutured to the body, a free edge not sutured to the body, and an inner surface;
wherein the inner surfaces of the first and second leaflets are adapted to coapt with each other along a catenary, the catenary being spaced from the free edges of the first and second leaflets and representing a line of interaction between the first and second leaflets nearest the cut edge.

17. The prosthetic valve of claim 16, wherein the first leaflet is cut from a first porcine aortic valve, and the second leaflet is cut from a second porcine aortic valve.

18. The prosthetic valve of claim 17, wherein the first and second leaflets are each a left cusp.

19. The prosthetic valve of claim 16, wherein upon closure of the prosthetic valve, the inner surface of the first leaflet redundantly coapts with the inner surface of the second leaflet.

20. (canceled)

21. The prosthetic valve of claim 16, wherein each of the free edges has a length that is longer than a length of the catenary.

22. The prosthetic valve of claim 16, wherein upon opening the prosthetic valve, the free edges define an opening having a perimeter greater than the twice a length of the catenary.

23. The prosthetic valve of claim 2016, wherein upon closure of the prosthetic valve, the first and second inner surfaces interact to define an enhanced surface area interface between the catenary and the free edges.

24. The prosthetic valve of claim 16, wherein the first and second leaflets each define a free edge not sutured to the body, and upon closure of the prosthetic valve, the free edges pucker.

25. The prosthetic valve of claim 16, wherein the first and second leaflets are coupled to the body in a manner characterized by a lack of tautness.

26. The prosthetic valve of claim 16, wherein the body includes a stent including an annular frame, a first strut extending from the annular frame, and a second strut spaced from the first strut and extending from the annular frame.

27. The prosthetic valve of claim 26, wherein the first strut and the second strut are nonsymmetrically positioned with respect to the annular frame.

28. The prosthetic valve of claim 16, wherein the prosthetic valve is a prosthetic mitral valve.

29. The prosthetic valve of claim 16, wherein the body includes a tubular body

30. The prosthetic valve of claim 29, wherein the tubular body is an aortic root.

31. A prosthetic valve comprising:
a body;
a first leaflet extending across and sutured to the body, the first leaflet being cut from a first porcine aortic valve, defining a first inner surface, and having an elongated shape; and
a second leaflet extending across and sutured to the body opposite the first leaflet, the second leaflet being cut from a second porcine aortic valve, defining a second inner surface, and having an elongated shape.

32. A method of manufacturing a prosthetic valve, the method comprising:
providing a body;
cutting a first leaflet defining a first inner surface from a first porcine aortic valve;
coupling the first leaflet to the body;
cutting a second leaflet defining a second inner surface from a second porcine aortic valve; and
coupling the second leaflet to the body opposite the first leaflet.

33. The method of claim 32, wherein the first leaflet and the second leaflet are each a left cusp.

34. The method of claim 32, wherein coupling the first leaflet and the second leaflet to the first and second struts includes positioning the first leaflet and the second leaflet upon the body such that the first and second inner surfaces redundantly coapt upon closure of the prosthetic valve.

35. The method of claim 32, wherein coupling the first leaflet and coupling the second leaflet to the first and second struts includes leaving a first free edge of the first leaflet and a second free edge of the second leaflet unsutured to the body, respectively, wherein the free edges are adapted to pucker upon closure of the prosthetic valve.

36. The method of claim 32, wherein cutting the first and second leaflets includes selecting the first and second leaflets from a plurality of porcine aortic valves previously harvested for potential use in a prosthetic aortic valve.

37. The method of claim 32, wherein cutting the first and second leaflets includes selecting the first and second leaflets from a plurality of leaflets, and each of the plurality of leaflets differs in size from each of the other plurality of leaflets.

38. The method of claim 37, wherein selecting the first and second leaflets includes selecting the first and second leaflets each having a size corresponding to a size of the body.

39. The method of claim 32, wherein the body includes a stent including an annular frame, a first stent extending from the annular frame, and a second strut extending from the annular frame spaced from the first stent.

40. The method of claim 32, wherein the body is a tubular body.
